(19) United States
(12) Patent Application Publication
(10) Pub. No.: US 2008/0255462 A1
(43) Pub. Date: Oct. 16, 2008

(54) LIGHT STICK

(75) Inventor: Mark Bride, Preoria, AZ (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: ZILA PHARMACEUTICALS, INC., Phoenix, AZ (US)

(21) Appl. No.: 12/101,023

(22) Filed: Apr. 10, 2008

Related U.S. Application Data
(63) Continuation-in-part of application No. 11/400,110, filed on Apr. 6, 2006.
Application No. 11/400,110 is a continuation-in-part of application No. 10/564,800, filed on Jan. 12, 2006.

(51) Int. Cl. A61B 6/00; C12Q 1/04; C12M 1/34

(52) U.S. Cl. 600/476; 435/34; 435/288.7

(57) ABSTRACT

Described herein is a device used to perform medical examinations. The device includes at least one sidewall and at least one end, wherein the sidewall at least partially defines both a chemical housing and a handle portion. The chemical housing preferably includes a light source disposed therein. A reflective layer may be applied to at least a portion of the sidewall defining the chemical housing.

### RELATED APPLICATIONS

This application is a continuation-in-part of application Ser. No. 11/400,110, filed Apr. 6, 2006, which is a continuation-in-part of application Ser. No. 10/564,800, filed Sep. 28, 2004, the contents of each of which are incorporated by reference in their entireties.

### FIELD OF THE INVENTION

The present invention relates generally to devices for detecting abnormal epithelial tissue, which may harbor tumor phenotypes.

BACKGROUND OF THE INVENTION

Patients who delay in obtaining a cancer consultation may have significantly higher relative hazards of death than do patients with a shorter delay. Thus, if patients are more regularly subjected to effective cancer screening, the mortality risks of cancer could be reduced. There is a need for a simple, rapid screening test for the detecting abnormal mucosal tissue which may harbor tumor phenotypes, which may indicate the presence of or the eventual development of invasive cancer, Abnormal epithelial tissue can be visually identified and located real time in vivo using selective light examination, which are admirably suited for rapid and inexpensive screening carried out as routine medical and dental examinations. Illustratively, U.S. Pat. Nos. 5,179,938 and 5,329,938 to Lonky, and U.S. Patent Application Publication Nos. 2006/0241494 and 2006/0241501 to Bride, the contents of each of which are incorporated herein by reference in their entireties, describe instruments equipped with a chemiluminescent light source which radiates in the visible green, blue, and optionally, red spectrums, with spectral peaks at 430, 550, and 580 nm. Under such illumination, with normal ambient light surpressed, abnormal mucosal tissue appears white. Illustratively, such selective light devices for practicing such in vivo examinations are commercially available under the registered trademark VIZILITE® from Zila Pharmaceuticals, Inc., Phoenix, Ariz., USA.

### SUMMARY OF THE PREFERRED EMBODIMENTS

According to a first aspect of the present invention, there is provided a device used to perform medical examinations. The preferred device includes at least one sidewall and at least one end. The sidewall at least partially defines both a chemical housing and a handle portion. The chemical housing preferably has an inside. The device further includes a reflective material or layer applied to at least a portion of the sidewall defining the chemical housing. Preferably, the chemical housing has a light source disposed therein.

According to another aspect of the present invention, there is provided a method for detecting abnormal epithelial tissue. The method preferably includes providing a device having at least one sidewall and at least one end. Preferably, the sidewall at least partially defines both a chemical housing and a handle portion. The chemical housing may have a light source disposed therein. The method further includes providing a reflective material or layer applied to at least a portion of the sidewall defining the chemical housing and illuminating an area of epithelial tissue with light emitted from the light source. Preferably, a portion of the light is reflected from the area thereby creating reflected light having at least one wavelength (referred to herein as “reflected light”). The method further includes viewing the reflected light.

According to another aspect of the present invention, there is provided a kit for medical examination. Preferably, the kit includes a device and a viewer such as spectacles having a filter. The device may include at least one sidewall and at least one end. The sidewall preferably defines at least a portion of both a chemical housing and a handle portion. A light source may be contained within the chemical housing. In a preferred embodiment, the kit further includes a reflective material or layer. The reflective material or layer is preferably applied to at least a portion of the sidewall defining the chemical housing.

### BRIEF DESCRIPTION OF THE DRAWINGS

The invention may be more readily understood by referring to the accompanying drawings in which:

FIG. 1 is a perspective view of a device in accordance with a preferred embodiment of the present invention;

FIG. 2 is a cross section of the device of FIG. 1 taken along the line 2-2 of FIG. 1;

FIG. 3A is a perspective view of the device of FIG. 1;

FIG. 3B is a side elevational view of one aspect of the device of FIG. 1; taken along line 3B/3C-3B/3C of FIG. 3A;

FIG. 3C is a side elevational view of another aspect of the device of FIG. 1 taken along the line 3B/3C-3B/3C of FIG. 3A;

FIG. 4 is a perspective view of the device in accordance with another embodiment of the present invention with a portion of a sidewall of the device removed;

FIG. 5 is a cross-section of the device of FIG. 4; taken along line 5-5 of FIG. 4;

FIG. 6 is a perspective view of the device in accordance with another embodiment of the present invention;

FIG. 7A is a cross-section of the device of FIG. 6; taken along line 7A/7B-7A/7B of FIG. 6;

FIG. 7B is another embodiment of the device of FIG. 6 taken along line 7A/7B-7A/7B of FIG. 6; and

FIG. 8 is a diagram of the light emitted from a light source.

Like numerals refer to like parts throughout the several views of the drawings.

### DETAILED DESCRIPTION OF THE PREFERRED EMBODIMENTS

As shown in the drawings, for purposes of illustration, the invention is embodied in an apparatus and method for medical examination (as used herein, may refer to “dental examination”). Preferably, the present invention is used to detect abnormal epithelial tissue (as used herein, tissue characterized as “abnormal” may include any/all of the following: pre-cancerous tissue, cancerous tissue; tissue experiencing angiogenesis; tissue exhibiting molecular/genetic signs of precancer/cancer; tissue having cells with abnormal apoptotic pathways, etc.). The apparatus preferably includes a light source (as used herein, “light source” may refer to any/all of the components of a chemiluminescence reaction). The light source preferably emits light. Preferably, the device includes a reflective layer (as used herein, may refer to “reflective material”) disposed thereon.

Briefly, one embodiment of the invention for screening epithelial tissue for possible abnormal tissue includes illuminating a gross anatomical area of epithelial tissue with the light emitted from the light source. Preferably, at least a portion of the light emitted from the light source is reflected off the reflective layer back into the chemical housing Additionally, at least a portion of the light emitted from the light source is incident light (as used herein, the incident light and light reflected off the reflective layer back into the chemical housing are collectively referred to as “emitted light”). However, the device is not limited to the detection of abnormal epithelial tissue and may detect other types of tissue/cells.

The emitted light selectively aids in visualizing abnormal tissue sites on the gross area. Abnormal tissue sites preferably appear white or substantially white. A filter lens may be used to view the illuminated gross area of tissue. Use of the filter lens allows the examiner to view reflected light only in certain desired wavelengths, while substantially blocking transmission of ambient or interfering light of wavelengths other than the reflected light of the desired wavelengths, thus enhancing the selective visualization of any abnormal tissue sites in the presence of normal ambient light.

For exemplary purposes only, described hereinbelow is a preferred embodiment wherein the device houses a chemliuminescent light source, 9,10 diphenylanthracene (“DPHA”). DPHA is used as a fluorescer in a peroxyoxalate chemiluminescence system (hydrogen peroxide is used as the energy source). However, this is not a limitation on the present invention. It will be understood that the method and device may use any other type of chemiluminescent light source, or any other light source, for example, incandescent, fluorescent, or the like. Any light source that emits incident light that can be reflected back in a selected spectral range is within the scope of the present invention. Other light sources will be readily apparent to those skilled in the relevant art.

It will be appreciated that terms such as “front,” “back,” “top,” “bottom,” “side,” and the like used herein are merely for ease of description and refer to the orientation of the components as shown in the figures. It should be understood that any orientation of the device, and the components thereof, described herein, is within the scope of the present invention.

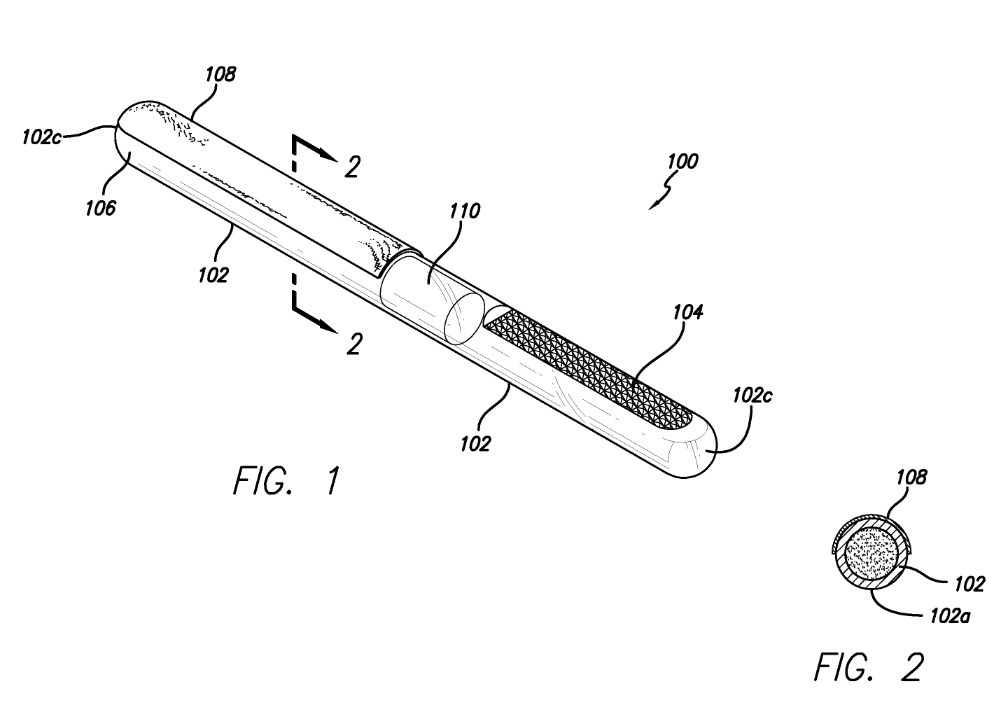

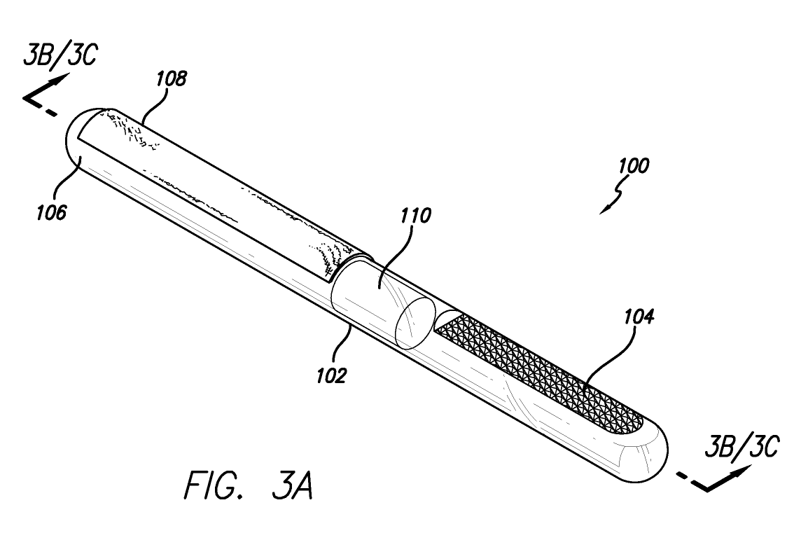

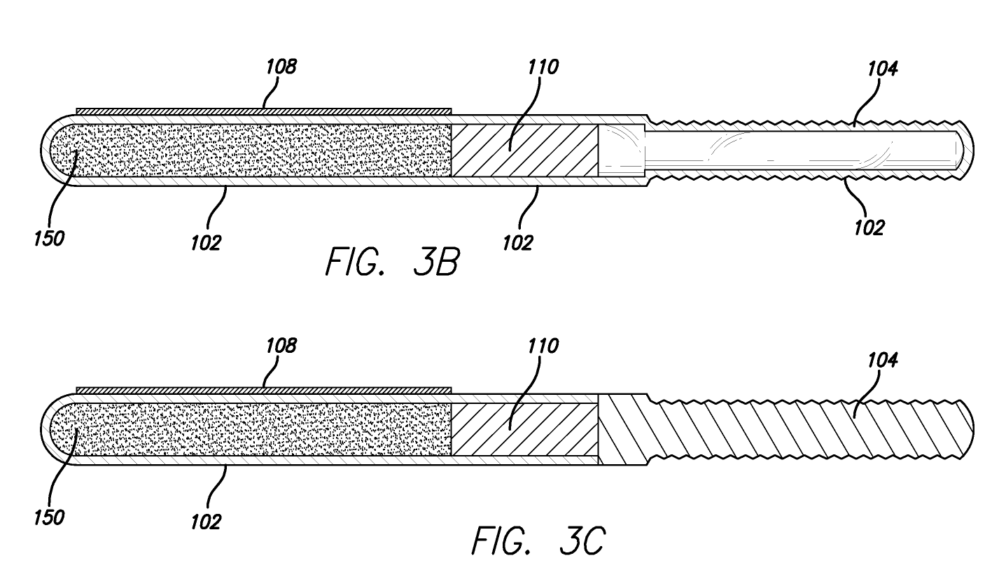

Generally, the present invention may be briefly described as follows. Referring initially to FIGS. 1-8, a device 100 used for medical examination is described. In a preferred embodiment, the device 100 is a self-contained, one-piece device. Preferably, the device 100 includes a sidewall 102, a chemical housing 106, a handle portion 104, and two ends 102c (referred to individually and collectively as “102c”). Accordingly, and as seen in FIGS. 1-5B, the device 100 is preferably tubular in shape. The sidewall 102 preferably defines both the entire chemical housing 106 and the entire handle portion 104. As such, the chemical housing 106 is preferably contiguous with the handle portion 104. As best seen in FIGS. 1 and 2, a reflective material or layer 108 is preferably applied to an outer surface 102a of at least a portion of the sidewall 102 defining the chemical housing 106. Preferably, the reflective material or layer 108 is a reflective tape. As best seen in FIGS. 3B-3C, the chemical housing 106 preferably includes a light source 150 disposed therein.

In other embodiments, the sidewall 102 may define the entire chemical housing 106 and a portion of the handle portion 104, or may define a portion of the chemical housing 106 and the entire handle portion 104. Likewise, in other embodiments, the sidewall 102 may define a portion of the chemical housing 106 and a portion of the handle portion 104. Accordingly, a portion of the chemical housing 106 and/or a portion of the handle portion 104 may be defined by another sidewall or other suitable component, without departing from the scope of the present invention.

The reflective layer 108 is preferably applied as follows. Referring now to FIGS. 1 and 2, the reflective layer 108 is preferably applied along the length of the sidewall 102 defining the chemical housing 106. Since the device 100 is preferably tubular in shape, a cross-section of the device 100 is circular or substantially circular and is therefore 360°. In a preferred embodiment, the reflective layer 108 is applied around about 50° to about 300° of the cross-section. In a more preferred embodiment, the reflective layer 108 is applied around about 100° to about 250° of the cross-section. In a highly preferred embodiment, the reflective layer 108 is applied around about 180° of the cross-section. Accordingly, the reflective layer 108 is preferably applied on about 50% of the circumference of the outer surface 102a of the sidewall 102. In other embodiments, the reflective layer 108 may be applied less than about 180° or more than 180° around the outer surface 102a of the sidewall 102 defining the chemical housing 106. Additionally, it is to be understood that the reflective layer 108 may be applied along the length as well as the width of the sidewall/or applied to the width alone, without departing from the scope of the present invention. It should be applied sufficiently to the sidewall 102 to provide the intended reflective effect.

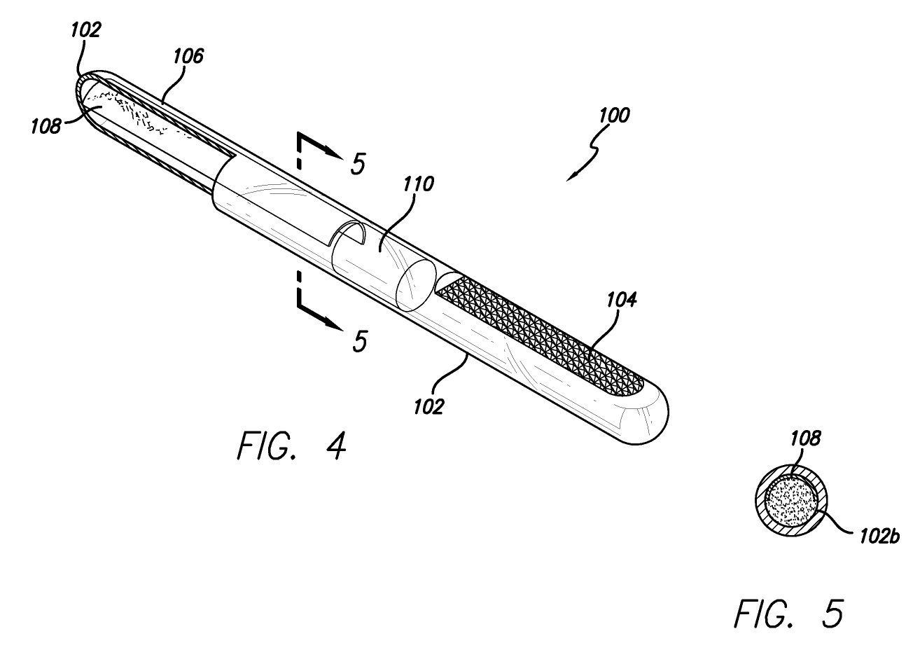

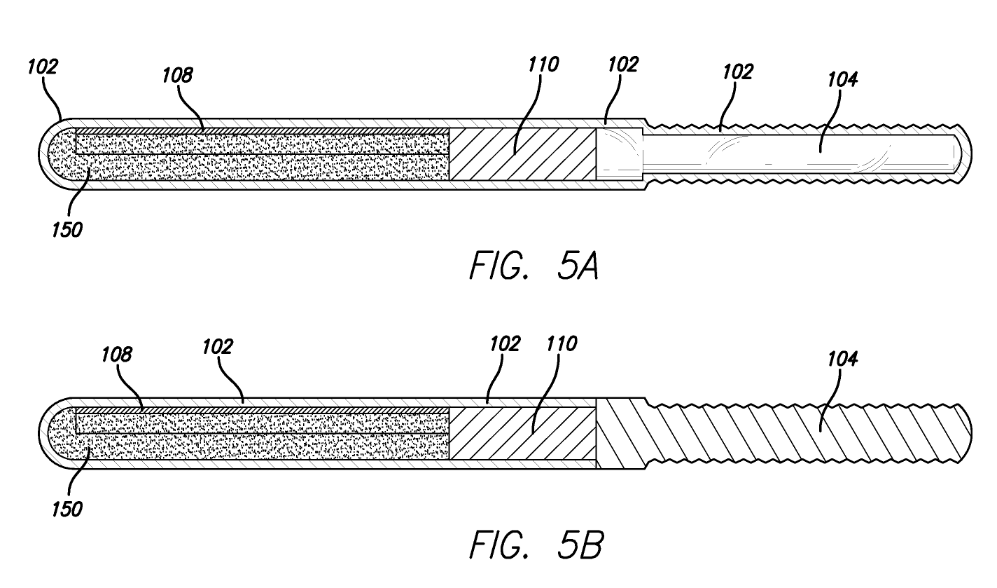

As best seen in FIGS. 4-5B, in other embodiments, the reflective layer 108 may be applied to an inner surface 102b of the sidewall 102 defining the chemical housing 106. As such, the reflective layer 108 is disposed on an inside of the chemical housing 106. A reflective layer 108 applied to the inner surface 102b operates similarly to a reflective layer 108 applied to the outer surface 102a: at least a portion of the light emitted from the light source 150 reflects off the reflective layer 108 back into the chemical housing 106 and at least a portion of the light emitted from the light source 150 is incident light. Accordingly, it is to be understood that the discussion that pertains to the reflective layer 108 applied to the outer surface 102a of the sidewall 102 also pertains to the embodiments wherein the reflective layer 108 is applied to the inner surface 102b.

Additionally, it is to be understood that the reflective material or layer 108 may be applied (as used herein, interchangeable with “disposed” or “printed”) to the sidewall 102 in any fashion and/or any configuration. Accordingly, the reflective material or layer 108 may include an adhesive on one or both of its sides, and/or or the sidewall 102 may include an adhesive on its outer and/or inner surfaces for attaching the reflective layer 108 to the sidewall 102. Additionally, more than one reflective layer 108 may be applied to the sidewall 102. When more than one reflective layer is used, the reflective layers may at least be partially disposed on top of each other, or may be applied in separate locations on at least a portion of the sidewall 102 defining the chemical housing 106. In other embodiments, the reflective layer 108 may be applied to at least a portion of the sidewall 102 defining the chemical housing 106 and at least a portion of the sidewall 102 defining the handle portion 104. Additionally, the reflective material 108 may be spotted onto the sidewall 102.

Preferably, the reflective layer 108 is comprised of plastic. Examples of plastics include, but are not limited to, polyethylene, polyvinyl chloride, polyvinylidene chloride, polypropylene, polyamide, polycarbonate, polytetrafluoroethylene, polyurethane, polystyrene, or the like. It is to be understood that the reflective layer 108 may be made out of any suitable material that has a high reflective index. Examples of such materials include polystyrene, styrene-methyl methacrylate copolymers, (meth)acrylic resin, polymethyl pentene, allyl glycol carbonate resin, spirane resin, amorphous polyolefin, polycarbonate, polyamide, polyallylate, polysulfone, polyallyl sulfone, polyether sulfone, polyether imide, polyimide diallyl phthalate, fluoro-resins, polyester carbonate, resin of norbornene family (ARTON), alicylic acrylic resin, silicone resin, acrylic rubber, and silicone rubber.

In a preferred embodiment, the thickness of the reflective layer 108 is from about 2 mm to about 0.02 mm. In a more preferred embodiment, the thickness of the reflective layer 108 is from about 1.0 mm to about 0.04 mm. In a highly preferred embodiment, the thickness of the reflective layer 108 is from about 0.07 mm to about 0.05 mm. However, the thickness of the layer may be more than 2 mm or less than 0.04 mm without departing from the scope of the present invention. Generally, when the reflective layer 108 is too thin, the efficiency of light reflection is reduced. Accordingly, the thickness of the reflective layer 108 should take this into account. As such, the reflective layer 108 is preferably of a thickness that maximizes the efficiency of light reflection and/or maximizes light output.

In a preferred embodiment, the sidewall 102 defining the chemical housing 106 is opaque or substantially opaque. This allows the emitted light to be transmitted to the outside. In other embodiments, any suitable material that allows the light source 150 to transmit light through at least a portion of the sidewall 102 defining the chemical housing 106 is within the scope of the present invention. For example, at least a portion of the sidewall 102 may be transparent or translucent. In some embodiments, the material of the sidewall 102 defining the chemical housing 106 may be chosen so as to maximize light output.

Referring now to FIGS. 1-8, and as best seen in FIG. 8, the device 100 preferably operates as follows. Preferably the chemical housing 106 is bent in order to activate the chemiluminescence reaction. Bending may mix the components of the chemiluminescence reaction in the following manner: The chemical housing 106 may be comprised of two compartments. A first (inner) compartment is breakable, such as a frangible ampoule, and contains one of the components, either the activator of the fluorescer. A second (outer) compartment (preferably defined by the sidewall 102) is partially or fully flexible, but is sturdy enough to resist being cut or broken when the ampoule is broken. It contains the other component. Accordingly, bending the sidewall 102 defining the chemical housing 106 at least partially breaks the inner compartment, exposing the contents therein to the contents of the outer compartment in the chemical housing 106, thereby initiating the chemiluminescence reaction. Accordingly, light is emitted from the reaction. In other embodiments, the light source 150 may be activated by other means. For example, a plunger may be used to mix one component with another in the chemical housing 106.

As best seen in FIG. 8, at least a portion of the light emitted from the light source 150 is reflected off the reflective layer 108 back into the chemical housing 106. The light reflected off the reflective layer 108 is represented by “310.”Additionally, a portion of the light emitted from the light source is incident light, which is represented by “320.” In a preferred embodiment, the light reflected off the reflective layer 310 and the incident light 320 together provide a light output that is greater than the light output of the incident light 320 alone. An additive effect of the light 310 reflected off the reflective layer 108 and the incident light 320 preferably provides this increase in light output.

Preferably, the light source 150 of the device 100 is directed to either the oral cavity or the vaginal cavity and preferably to the tissue/cells of interest. As such, the portion of the sidewall 102 not covered by the reflective layer 108 preferably faces the tissue of interest. The emitted light is thus directed to the tissue/cells of interest. Abnormal tissue generally appears different than normal tissue. Abnormal tissue appears white or substantially white, and regular tissue does not appear white. The tissue/cells of interest may be viewed in the dark or in ambient light preferably through a filter lens.

The size of the device 100 may vary depending upon its intended use. Tn a preferred embodiment, the size of the chemical housing 106 is large enough to contain a sufficient amount of chemiluminescent material to light at least a portion of the cavity to be examined. The device should be of a size that is capable of being disposed within the cavity adjacent the area of the cavity to be examined. Accordingly, a device suitable for vaginal use may be smaller than a device suitable for oral use.

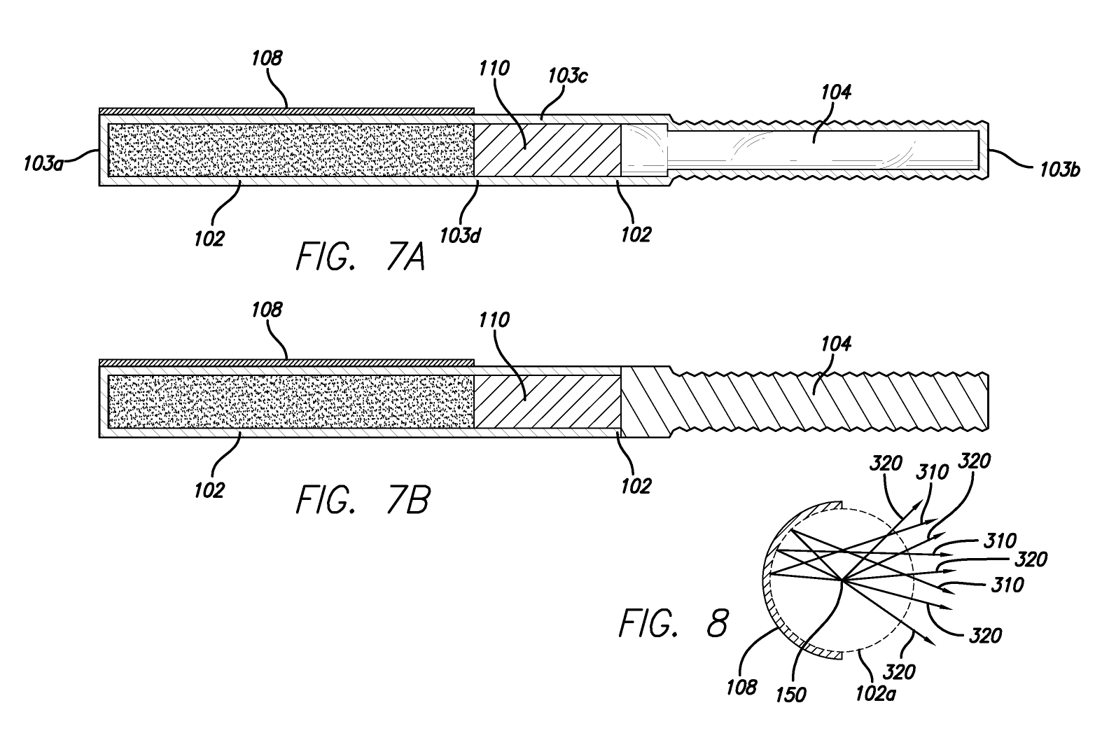

In a preferred embodiment, and as seen in FIGS. 5A and 7A, the handle portion 104 has an inside. However, the handle portion 104 may not have an inside, as seen in FIGS. 5B and 7B. Additionally, in a preferred embodiment, the sidewall 102 defining the handle portion 104 is opaque or substantially opaque. In other embodiments, the handle portion 104 may be made of any suitable material. The material may or may not be able to transmit light.

Additionally, in some embodiments, a stop deposit 110 may be disposed between the light source 150 and the handle portion 104. The stop deposit 110 is preferably defined by the sidewall 102. It is preferably used to separate the light source from the handle portion 104. The stop deposit 110 may be made out of wax or paraffin; however, any material that does not absorb, or only partially absorbs, the light source 150 (i.e., the chemicals within the light source) is within the scope of the present invention. In other embodiments, the stop deposit 110 may be omitted or may be replaced by another suitable structure.

Additionally, it is to be understood that the device of the present invention may be stored for long periods of time without loss of function, as the light source is not activated unless the chemical components are mixed together.

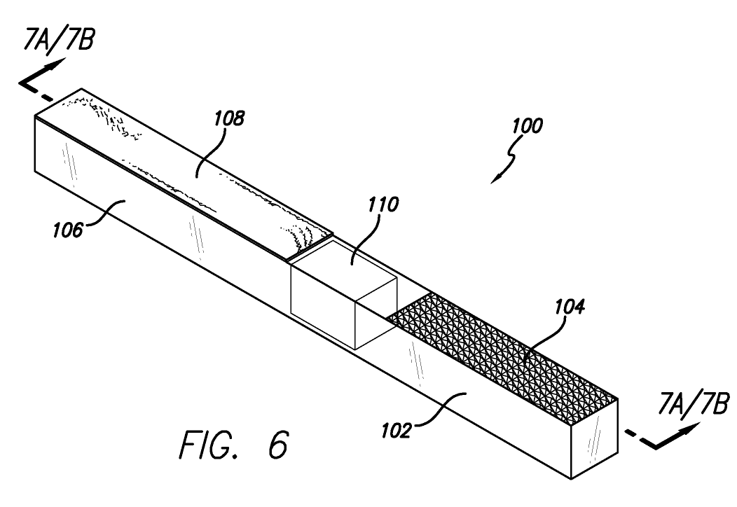

Referring now to FIGS. 6-7B, in other embodiments, the device 100 may be rectangular in shape. A device 100 that is rectangular in shape generally has four sidewalls (as seen in FIG. 7A, represented individually by 103a-d and collectively by 102). In a preferred embodiment, the reflective layer 108 is applied from about 10% to about 85% of the outer surface of the sidewall 102. In a more preferred embodiment, the reflective layer 108 is applied from about 25% to about 70% of the outer surface of the sidewall 102. In a highly preferred embodiment, the reflective layer 108 is applied to about 50% of the outer surface of the sidewall 102. In other embodiments, the reflective layer 108 may be applied to more than 50% or less than 50% of the outer surface of the sidewall 102.

In another embodiment of the present invention, there is provided a kit for medical examination. The kit preferably includes the device of the present invention as well as a light source contained therein. The device preferably includes a reflective tape applied thereon. Spectacles having a filter are preferably included in the kit.

The following examples are presented to enable those skilled in the art to understand and practice the invention and to identify the presently preferred embodiments thereof. These examples are provided for illustrative purposes and not to indicate the scope of the invention which is defined only by the appended claims.

### EXAMPLE 1

A routine visual examination of the oral cavity is made, noting the presence or absence of any lesions on the attached gingival, the buccal mucosa, the floor of the mouth, the hard and soft palate, and the dorsal, lateral, and ventral tongue. The presence or absence of any lesions noted by this routine examination are recorded. Additionally, the presence or absence of clusters of blood vessels (i.e., angiogenesis) which may indicate new growth such as cancer is noted.

### EXAMPLE 2

After completing the routine examination of Example 1, the patient is then instructed to rinse the mouth with a 1% acetic acid solution for up to one minute and then expectorate. Referring to FIG. 1, the device 100 of the present invention is activated by bending the sidewall 102 defining the chemical housing 106, thereby allowing the components therein to mix together.

Preferably, and as indicated earlier, 9,10 diphenylanthracine (“DPHA”) is used as the light source, and the light provided has a spectral peak of about 470 nm. This spectral peak preferably produces a blue light. In a preferred embodiment, use of DPHA reduces the amount of mucosal glare and provides a softer light than the use of other chemiluminescent agents. In other embodiments, the chemiluminescent light source described in U.S. Pat. No. 5,329,938 to Lonky, the entire contents of which are herein incorporated by reference, may be used. The light source described in that patent is commercially available under VIZILITE. The light provided has spectral peaks at about 430 nm, 550 nm, and a smaller peak in the red region at about 600 nm, and the spectral peaks produce a bluish-white light.

Tn a preferred embodiment, the examining clinician then dons a pair of spectacles provided with at least one lens which only transmits light in the wavelength band of 400-600 nm. These spectacles are shaped to minimize illumination reaching the examiner's eyes from above and from the sides. For example, the spectacles as disclosed in U.S. Patent Application Publication No. 2006/0241494 to Bride, the contents of which are herein incorporated by reference in their entirety, may be used.

Without reducing ambient light from normal illumination sources, the visual examination of the oral cavity is then repeated using the illumination provided by the light source, looking for lesions or other suspect tissue sites that appear white, paying attention to any suspect tissue sites noted in the routine examination of Example 1. Any sites which appear white or bluish white are noted and recorded.

Further assessment of the noted sites is made, for example by tissue biopsy for standard histology or by molecular analysis, to determine whether the tissue is cancerous or harbors mutations which are in the pathway for eventual development of invasive cancer. Molecular analysis may include DCR, such as microsatellite analysis, or the like.

### EXAMPLE 3

After completing the routine examination of Example 1, the patient is then instructed to rinse the mouth with a 1% acetic acid solution for up to a minute and then expectorate. Referring to FIGS. 1 and 2, the light source 150 of the device 100 of the present invention is then activated by bending the sidewall 102 defining the chemical housing 106.

The light source 150 emits light when activated. A portion of this light reflects off the reflective layer and travels back into the chemical housing. Preferably, at least a portion of this light is transmitted through the sidewall 102 defining the chemical housing 106. Additionally, a portion of the light emitted is incident light (as indicated earlier, collectively referred to herein as “emitted light”). Preferably, the light provided has a spectral peak at about 470 nm.

The emitted light is directed to the suspected pre-cancerous or cancerous region. The device is preferably manipulated so that little or none of the emitted light is directed toward the eyes of the examiner before being reflected. The examination is performed looking for lesions or other suspect tissue sites which appear white, paying attention to any suspect tissue sites noted in the routine examination of Example 1. Any sites which appear white or bluish-white are noted and recorded.

### EXAMPLE 4

A routine medical examination of the vaginal cavity, such as a routine pelvic exam, is conducted using an endoscope and/or a speculum. For example, a visual examination of the vaginal cavity is conducted, noting the presence or absence of any lesions, irregular vascularature, exophytic regions, ulcerations, and other aptypias of the cervix and vaginal cavity. A speculum may be used to spread apart the vaginal walls, allowing the inside of the vagina and cervix to be examined. The speculum may be plastic, metal, or any other material. The presence or absence of any lesions noted by this routine examination is recorded.

### EXAMPLE 5

The vaginal mucosal membrane is then coated with a solution of 1% to 10% acetic acid. The device of the present invention is then bent in order to mix the components of the light source together. In a preferred embodiment, the light provided has a single spectral peak at about 470 nm. This spectral peak produces a bluish light.

In a preferred embodiment, the examiner then dons a pair of spectacles provided with at least one lens which only transmits light in the wavelength band of about 450-500 nm.

A speculum or other suitable structure/endoscope is used to hold the vaginal cavity open. The device is then inserted into the vaginal passage so that the light source is directed to the tissue of interest in the vaginal cavity. It is to be understood that the speculum may be disposable or may be sterilized after use. Without reducing ambient light from normal illumination sources, the visual examination of the oral cavity is then repeated using the illumination provided by the light source, looking for lesions or other suspect tissue sites which appear white, paying attention to any suspect tissue sites noted in the routine examination of Example 1.

Further assessment of the noted sites is made, for example by tissue biopsy for standard histology or by molecular analysis, to determine whether the tissue is cancerous or harbors mutations which are in the pathway for eventual development of cancer. Molecular analysis may include PCR and/or microsatellite analysis.

It will be understood that the present invention can be used for examination of other areas of the body, besides the oral or vaginal cavities. For example, the present invention can be used to perform an endoscopic examination of the esophagus, the cervix, or the colon, to detect early signs of cancer. In this type of examination, a sleeve or the like with an opening for emitting light is inserted into the patient's esophagus, cervix, or colon and the examination is performed. As will be understood by those skilled in the art of endoscopy, the inside of the organ and the reflected light is viewed remotely on a monitor.

The embodiments described above are exemplary embodiments of the present invention. Those skilled in the art may now make numerous uses of, and departures from, the above-described embodiments without departing from the inventive concepts disclosed herein. Accordingly, the present invention is to be defined solely by the scope of the following claims.

What is claimed is:

1. A device used to perform medical examinations comprising:
(a) at least one sidewall and at least one end, wherein the sidewall at least partially defines both a chemical housing and a handle portion; wherein the chemical housing has an inside; and
(b) a reflective material applied to at least a portion of the sidewall defining the chemical housing;
wherein the chemical housing comprises a light source disposed therein.

2. The device of claim 1, wherein the sidewall comprises an outer surface, and wherein the reflective material is a layer applied to at least a portion of the outer surface of the sidewall defining the chemical housing.

3. The device of claim 2, wherein the reflective layer is applied to at least a portion of a length of the outer surface of the sidewall defining the chemical housing.

4. The device of claim 1, wherein the light source is a chemiluminescent.

5. The device of claim 1, wherein the device is suitable for oral or vaginal use.

6. The device of claim 1, further comprising a stop deposit disposed between the chemical housing and the handle portion.

7. The device of claim 1, wherein the device is used to detect abnormal epithelial cells.

8. The device of claim 1, wherein the sidewall defining the chemical housing is at least partially opaque.

9. A method for detecting abnormal epithelial tissue comprising:
(a) providing a device having at least one sidewall and at least one end, wherein the sidewall at least partially defines both a chemical housing and a handle portion; wherein the chemical housing has a light source disposed therein;
(b) providing a reflective material applied on at least a portion of the sidewall defining the chemical housing;
(c) illuminating an area of epithelial tissue with light emitted from the light source, wherein at least a portion of the light is reflected from the area thereby creating reflected light having at least one wavelength, and viewing the reflected light.

10. The method of claim 9, wherein the sidewall defining the chemical housing is at least partially opaque.

11. The method of claim 10, further comprising the step of determining if the reflected light is white.

12. The method of claim 10, wherein at least a portion of the light emitted from the light source is reflected off the reflective material back into the chemical housing and wherein at least a portion of the light emitted from the light source is incident light.

13. The method of claim 9, further comprising the step of bending the device to activate the light source prior to the step of (c).

14. The method of claim 10, further comprising the step of filtering the reflected light to substantially remove wavelengths other than the at least one wavelength, thereby creating filtered light, and viewing the filtered light.

15. The method of claim 14, further comprising the step of providing spectacles having a filter, and wherein the step of filtering the reflected light comprises filtering the reflected light with the spectacles to only allow filtered light through.

16. A kit for medical examination comprising:
(a) a device comprising:
(i) at least one sidewall and at least one end, wherein the sidewall defines at least a portion of both a chemical housing and a handle portion;
(ii) a light source contained within the chemical housing; and
(b) spectacles having a filter.

17. The kit of claim 16, further comprising a reflective material forming a reflective layer, wherein the reflective layer is applied to at least a portion of the sidewall defining the chemical housing.

18. The device of claim 1, wherein the light source is 9,10 diphenylanthracene.

19. The device of claim 8, wherein the light source provides a light output, and wherein the light output comprises incident light and light reflected off the reflective layer back into the chemical housing.

20. The method of claim 15, further comprising the step of performing an assessment of the area, wherein the assessment of the area is selected from the group consisting of a tissue biopsy, a histological analysis, or a molecular analysis.

21. The device of claim 1, wherein the reflective material is a reflective tape.
